(19) United States
(12) Patent Application Publication
(10) Pub. No.: US 2010/0145224 A1
(43) Pub. Date: Jun. 10, 2010

(54) CANAL DILATION DEVICE

(75) Inventors: Stephen Lee, Glen Iris (AU); Jason Jit-sun Tan, Hamilton (AU)

Correspondence Address: HOVEY WILLIAMS LLP, 10801 Mastin Blvd., Suite 1000, Overland Park, KS, 66210, US

(73) Assignee: LEVEL THREE INNOVATIONS PTY LTD, Glen Iris, Victoria (AU)

(21) Appl. No.: 12/524,340

(22) PCT Filed: Jan. 25, 2008
(86) PCT No.: PCT/AU08/00082

§ 371 Date: Jan. 28, 2010

(30) Foreign Application Priority Data
Jan. 25, 2007 (AU) 2007900380

(51) Int. Cl. A61M 29/02; A61B 10/02

(52) U.S. Cl. 600/562; 606/193

(57) ABSTRACT

A device for enlarging a canal of a human or animal subject includes an elongate member having a first lumen extending therein and an inflatable body toward a distal end of the elongate member. The inflatable body is deliverable, in an uninflated or deflated state, to a location substantially within the canal prior to inflation. The first lumen is in communication with the inflatable body via one or more channels therebetween. In use, the first lumen is in communication, toward a proximal end, with an inflation system for delivering fluid to inflate the inflatable body and thereby enlarging the canal. The device may include markings for sounding of e.g. uterine depth. The device may also include one or more projections, or other structure providing tactile cues during positioning of the device within the canal and these may also be configured to aid in removal of cells from a tissue wall.

### FIELD OF THE INVENTION

The present invention relates to a device for dilating a canal, such as a cervical canal or urethra, of a human or animal subject. The invention also relates to a method and system for doing the same.

### BACKGROUND TO THE INVENTION

Dilation of the cervix and cervical canal is performed in procedures where access to and/or instrumentation of the interior of the uterus is required. Dilation typically involves use of Hagar dilators, a set of curved stainless steel rods each having a different cross-sectional diameter ranging from 2 Hagar (2 mm) to 14 Hagar (14 mm). To dilate the cervix, a small Hagar dilator is inserted through the external cervical os and into the cervical canal. It is then removed and a larger dilator is inserted. This process is repeated using progressively larger diameter dilators until the desired amount of cervical dilation is achieved.

Dilation of the cervix is commonly accompanied by sounding of the uterus to determine uterine depth and to confirm the position of the uterus. Uterine sounding typically involves insertion of an elongate, curved instrument having a blunt distal tip into the cervix. The sound has a proximal handle and markings, usually in centimetres, etched along the entire length of the sound. Prior to an intrauterine procedure, the distal end of the sound is inserted into the cervical canal and the physician reads the length marking that is visible just exterior to the external cervical os to sound the uterine cavity.

Whilst use of Hagar dilators and metal uterine sounds is common in gynaecological procedures, use of these instruments is necessarily forceful as dilation of the cervical tissue is achieved by gradually tearing the cervical tissue fibres. Repeated cervical entry using Hagar dilators of increasing size is associated with cervical trauma, tears and bleeding as larger dilators are forced through the cervical canal. Also, use of Hagar dilators and cervical sounds is associated with accidental uterine perforation. This is attributable in part, to the forcefulness with which the instruments must be used, and is also due to their pointed structure and rigidity. Uterine perforation can result in complications such as haemorrhage and infection, sometimes necessitating additional procedures such as laparoscopy and laparotomy to correct the complications.

It has been reported that uterine perforation occurs in at least 1.98% of dilation and curettage cases. This equates to more than 19.8 people in 1000 (Kaali S G, Szigetvari I A, Bartfai G S “The frequency and management of uterine perforations during first-trimester abortions” American Journal of Obstetrics & Gynecology 1989 August; 161(2):406-8). These statistics do not include uterine perforation resulting from other higher-risk uterine procedures requiring cervical dilation such as hysteroscopy or other instrumentation of the uterus when intrauterine pathology is present.

It would be desirable to provide an alternative instrument for dilating the cervix.

The discussion of the background to the invention included herein including reference to documents, acts, materials, devices, articles and the like is intended to explain the context of the present invention. This is not to be taken as an admission or a suggestion that any of the material referred to was published, known or part of the common general knowledge in the patent area as at the priority date of any of the claims.

### SUMMARY OF THE INVENTION

According to a first aspect of the present invention, there is provided a dilation device for enlarging a canal of a human or animal subject, the device including: an elongate member having a first lumen extending therein and an inflatable body toward a distal end of the elongate member. The inflatable body is deliverable to a location substantially within the canal prior to inflation. The first lumen is in communication with the inflatable body via one or more channels therebetween and when in use, is also in communication, toward a proximal end, with an inflation system for delivering fluid to inflate the inflatable body and thereby enlarge the canal.

Preferably, the canal being enlarged is the cervical canal and for the purpose of describing the invention and its various embodiments, use of the device in the cervical canal will be described. However, it is to be understood that the device, method and system of the present invention is useful for dilating or sealing other canals of the body, such as the urethra for example, and for accessing or draining cavities e.g. the bladder, located at a distal end of the canal. The invention may also be used as a drainage tube for exiting fluid from cavities within the body. In addition, the present invention may be utilised in the deployment of ureteric or other stents locatable in the canal. Once the tip of the device is positioned within a cavity distal of the canal (e.g. the uterus or bladder), a stent or similar object may be delivered through a device lumen and introduced into the ostia opening into the cavity of e.g. the uterus or ureteric orifices of the bladder.

Preferably, when the inflatable body is in a deflated state, the maximum diameter of the part of the device inserted into the cervical canal is 2-3 mm. This enables the device to be delivered to the cervix without use of a sheath, although it is to be understood that overall diameters of 3.5 mm, 4 mm, 4.5 mm or 5 mm may also be suitable in some subjects. The inflatable body may be wrapped around or folded or crumpled onto the elongate member and retain its minimal size during delivery or deployment into the cervical canal by use of a biocompatible adhesive or agent designed to fail when moistened (e.g. by the mucous in the cervix) and/or when warmed to body temperature (e.g. after insertion) and/or when inflated and/or when an electromagnetic field is present. Other means may be employed to keep the inflatable body in a form which gives the device a minimal overall diameter during insertion.

Alternatively/additionally, a delivery sheath may be provided to aid and guide the elongate member during insertion. After the inflatable body has been inserted and placed in position, the delivery sheath may be carefully retracted and removed from the subject.

In a preferred embodiment, the device includes markings along a length of the elongate member. The markings indicate a depth of insertion of the elongate member into the cervical canal and/or uterine cavity for use in uterine sounding. The markings may extend along a length of the elongate member beginning just inside the external cervical os when the elongate member has been inserted into the cervical canal and extending proximally. Alternatively, the markings may extend along a length of the elongate member, beginning at the distal tip of the device. The markings may extend along a defined length of the member or alternatively, along the entire length of the member. Preferably, the markings extend approximately 10 cm, 12 cm or 14 cm from the distal tip of the elongate member to facilitate sounding of the uterus during use although these dimensions are not to be taken as limiting.

The markings are useful for sounding the uterus to ascertain the size of the uterine cavity. This measurement is made routinely by gynaecologists for clinical purposes. Using the present invention, the sounding process involves inserting the device in an uninflated or deflated state so it may pass into the fundus/upper limit of the uterus until resistance is felt, indicating the distal wall of the cavity has been reached. Thus, the cavity/uterus and thus size will be indicated by the markings on the elongate member.

Where distal projections are provided, they extend outwardly once the uterus is entered. The device is then withdrawn and when the projections abut the cavity wall around the cervical os, they will meet a resistance while resting against the internal limit of the cervix. With the device so positioned, markings on the elongate member may be used to indicate the length of the cervix by calculating the difference between the reading of the markings from the total depth measured during sounding. This can also act as a guide for placement of the device in the canal, ensuring that the inflatable portion of the device is within the cervical canal which requires dilation.

The device may also include one or more collapsible projections located at or near the distal end of the device, distally of the inflatable body. The projections may, together, form an arrowhead shape toward the distal end of the elongate member when collapsed, to minimise the diameter of the device during insertion through the cervical canal. Upon passing the internal cervical os and entering the uterine cavity, the one or more projections assume an expanded state which, when a clinician gently draws the device proximally, provides a haptic cue felt by the clinician as the outwardly biased projections are drawn into abutment with the uterine wall around the internal cervical os. This haptic cue indicates that the inflatable body is located within the cervical canal.

Preferably, the collapsible projections are outwardly biased such that, when the distal end of the device has entered the uterine cavity, the projections expand or extend outwardly automatically and when drawn into abutment with the uterine wall around the internal cervical os, give the clinician a tactile cue as to correct placement of the inflatable body. Alternatively, the projections may be extended by rotating the device clockwise or counter-clockwise (and rotating counter-clockwise or clockwise to collapse them), or using an actuating means to expand and collapse the projections as required. Where outwardly biased projections are provided, to remove the device the projections may be sufficiently flaccid that they can fold backward as the device is drawn out of the cervical canal.

It is to be understood that one or more collapsible projections may take any suitable form. In another arrangement, the one or more projections are provided in the form of a collapsibly expandable framework located at or near the distal end of the elongate member and adapted to pass through canal in a collapsed state. Upon passing into the uterus, the framework assumes an expanded state by way of an outward bias. Shape memory materials may be used in the fabrication of such projections. To remove the device, a slight pull on the elongate member causes the framework to collapse upon itself enabling it to be withdrawn from the uterus and cervical canal.

One or more of the projections may include one or more special edges adapted to remove or “sample” cells from the uterine wall. The sampling edge may extend along only a portion of the one or more projections, or may cover all or most of the length of the one or more projections. The sampling edge or parts thereof may have a higher coefficient of friction than other regions of the projections, and/or may have a sharpened profile to aid collection of cells, debris or other matter from the uterine wall. Alternatively, the sampling edge may be used to agitate fluid in the uterus (e.g. by rotation of the device in situ) to slough cells off the uterine wall for biopsy or testing. The cells may adhere to the sampling edge, or they may be withdrawn from the uterus via a lumen in the elongate member.

In a preferred embodiment, the inflatable body has a substantially cylindrical shape when inflated to achieve uniform dilation of the cervical canal. However, conical, hourglass, round or oblong shapes are also contemplated, and may be selected to suit the anatomy and/or a condition of the subject's cervical canal. Alternatively or additionally, the inflatable body may be adapted to conform to the natural contours of the cervical canal when inflated.

In one arrangement, the elongate member further includes a second lumen for providing intrauterine access from outside the external cervical os while the cervix is being dilated (i.e. while the inflatable body is inflated within the cervical canal). In this embodiment, the device may also include means, such as for example, a tap, luer connector or fluid inlet/outlet for introducing a fluid into and withdrawing a fluid out of the uterine cavity via the second lumen. The fluid may include, for example, one or more of air, liquid, saline, contrast solution, antibiotic, coagulant, or other therapeutic agent.

The device may also include means for introducing an instrument into the uterine cavity via the second or a third lumen in the elongate member. The instrument may be selected from the group including but not limited to: illumination means (e.g. fibre-optic illuminator, LEDs) and visualisation means (e.g. miniaturised camera, CCD arrays); surgical, electrosurgical and laser tools; biopsy or other devices. Alternatively/additionally, implantable devices such as stents, staples and sutures may be deployed through the second or third lumen.

The inflatable body may be manufactured from any material possessing the necessary material qualities required to meet the inflation pressures and size limitations of the device. In one embodiment, it is preferable that the inflatable body is manufactured from a biocompatible material which is inextensible beyond a pre-defined shape and/or size.

The distal tip of the elongate member may be adapted to make a traumatic contact with tissue of the cervix or uterus during insertion and uterine sounding. Thus, the distal tip of the elongate member is preferably rounded and may include a soft and/or low friction coating to minimise risk of tissue trauma during insertion of the device and in particular, sounding of the uterus.

Preferably, the inflated body is adapted to form a seal in the canal, thereby substantially precluding leakage of fluids from the uterine (or other) cavity during, for example, hydrodilation of a fallopian tube. This procedure involves administering a fluid such as a dye, under pressure, into the uterus to determine, for example, fallopian tube patency. Inflation of the inflatable body to form a seal in the cervical canal substantially precludes leakage of such fluids from the uterine cavity.

The device may be adapted for use with an inflation system which, in one example, includes a motor-driven pump under the control of an automated control system. The inflation system is thereby adapted to deliver e.g. gradually increasing pressure to the inflatable body, controlling the rate and amount of canal dilation. Preferably, the control system includes one or more sensors to determine e.g. pressures within the inflatable body and/or a lumen in the elongate member, and the degree of cervical dilation/enlargement of the canal. The control system may also be adapted, when a pressure or cervical dilation above a predefined threshold is detected by the one or more sensors, to cause release of pressure from within the inflatable body.

In one embodiment of the present invention, the inflatable body is movable along a portion of the elongate member. The portion of the elongate member along which the inflatable body may move may be limited by stop members or protrusions on the external surface of the elongate member which prevent free-sliding of the inflatable body. When the inflatable body is inflated to a point where canal dilation occurs, further longitudinal movement of the inflatable body may be substantially precluded by frictional forces between the inflatable body and the elongate member and the protrusions on the elongate member.

Alternatively, one or more membranes may connect the inflatable body and the elongate member, permitting a degree of longitudinal movement of the inflatable body relative to the elongate member prior to and during early stages of inflation. When the inflatable body is inflated to a greater degree, the membranes are forced to angulate out from the elongate member as the body enlarges, thereby reducing the available longitudinal movement.

The elongate member may extend through and distally of the inflatable body. Thus, in one embodiment, the distal tip may contact a wall of the uterus (e.g. the uterine fundus) while the inflatable body is substantially within the cervical canal, although this need not be the case.

According to a second aspect of the present invention, there is provided a method for increasing the diameter of a canal of a human or animal subject, the method including the steps of: inserting a device for enlarging the canal, as described above, into the canal until the inflatable body is substantially within the canal in an uninflated or deflated state; and inflating the inflatable body by delivering fluid into the first lumen, thereby increasing the diameter of the canal to a required level of dilatation.

The method may include the additional step of sounding a chamber located distally of the canal, such as, in the case of increasing the cervical canal, the uterus. In this method, prior to inflation of the inflatable body the device is gently inserted until the distal tip of the device contacts uterine wall. This can be determined by the clinician by feel. The device includes markings, preferably graduated markings along a length of the elongate member which are used to measure the depth and/or position of the uterus. After insertion of the device, the clinician takes a reading using the markings, and these are indicative of the uterine depth sounded. The markings may be in millimetres, centimetres, inches or any other scale which is meaningful to the clinician and others involved in the procedure(s) being performed on the subject.

The method may also involve hydrodilation of a fallopian tube. In this method, the cervical canal is enlarged as described above, and the inflatable body forms a seal within the cervical canal. A fluid is then administered to the uterine cavity via a second lumen in the elongate member, to pressurise the uterine cavity. This method can also be used to administer dyes and other fluids to within the uterine cavity.

The seal formed by the inflatable body overcomes difficulties associated with other methods of hydrodilation in which fluid leakage is common at the catheter-cervix interface. Embodiments of the present invention provide a useful alternative approach to fallopian tube hydrodilation and other diagnostic tests of fallopian tube patency.

The method may also involve use of the device to extract cells from a cavity located distal of the canal (e.g. the uterus). This may involve use of the device to agitate cells to remove them from the cavity wall, prior to removal through a lumen of the device for pathology testing.

Another aspect of the present invention provides a system for dilating a canal of a human or animal subject. The system includes an enlarging device including an elongate member having a first lumen extending therein and in fluid communication with an inflatable body located toward a distal end of the elongate member, the inflatable body being deliverable, in an uninflated or deflated state, to a location substantially within the canal prior to inflation. Also provided is inflation means in fluid communication with the first lumen, for delivering fluid to inflate the inflatable body and achieve enlargement of the canal.

The device may further include markings along a region of the elongate member extending outside the external opening of the canal during use of the device, the markings indicating a depth of insertion of the member into the canal and/or a chamber located distally of the canal.

The device may further include one or more collapsible projections located at or near the distal end of the elongate member, the one or more projections adapted to pass through the canal in a collapsed state and, upon passing into a distal chamber, assuming an expanded state. Preferably, the one or more collapsible projections are outwardly biased. The one or more collapsible projections may further include a sampling edge adapted for removal of cells or matter from within a chamber distal located distally of the canal.

### BRIEF DESCRIPTION OF THE DRAWINGS

The present invention will now be described in greater detail with reference to the accompanying drawings. It is to be understood that the embodiments illustrated in the accompanying drawings are examples only and do not supersede the generality of the preceding description of the invention.

FIG. 1a is a cross-sectional view of a device, according to an embodiment of the present invention, with the inflatable body in a deflated state.

FIG. 1b shows the device of FIG. 1a in situ within the cervical canal, with the inflatable body inflated; it also shows in broken lines, the device being used to sound the uterus, with the inflatable body in a deflated and minimised state.

FIG. 2 shows an alternative embodiment of the present invention which incorporates collapsible projections used as a guide for locating the device within the cervical canal.

FIG. 3a shows another alternative embodiment of the invention which incorporates collapsible projections in the form of a collapsibly expandable framework.

FIG. 3b illustrates the embodiment illustrated in FIG. 3a with the collapsible projections in the expanded state within the uterus.

FIG. 4 shows a cross-section of another embodiment of the invention in which a second lumen is provided within the elongate member to provide intra-uterine access during cervical dilation.

### DETAILED DESCRIPTION OF THE INVENTION

The anatomy of the cervix (including the cervical canal) varies considerably between females of different ages and before and after natural childbirth. The endocervical canal (hereinafter referred to as the cervical canal) which is the passageway between the external cervical os (which projects into the vagina) and internal cervical os (which projects into the uterine cavity) varies in length and width although it is typically 5 mm to 10 mm wide and 20 mm to 50 mm long in women.

Enlarging or increasing the cervical canal is also known as “cervical dilation” and it is often necessary prior to performing a range of intrauterine procedures such as curettage, hysteroscopy, biopsy, termination of pregnancy, visualisation of the uterus, and dilation of the fallopian tubes. Other procedures in which cervical dilation may be required include placement or removal of intrauterine devices, induction of labour and endometrial biopsy to name a few. The present invention provides a new device and method for dilating the cervix and which, in various embodiments, may also be used for uterine sounding and/or hydrodilation of the uterus and fallopian tubes. These embodiments will now be described by reference to the drawings. Where possible, like numerals have been used to identify like features of the invention.

For the purpose of describing the invention and its various embodiments, use of the invention in the cervical canal will be described. However, it is to be understood that the device, method and system of the present invention is suitable for use in other canals of a human or animal subject's body.

Throughout the description and claims, the terms “proximal” and “distal” are used. These terms are determined by reference to a notional clinician who is treating a subject using the inventive device, where “proximal” refers to a region closer to the clinician and “distal” refers to a region further from the clinician. For example, when the device is moved proximally within the cervical canal, it is drawn toward the clinician whereas if it is moved distally, it is inserted into the canal, moving away from the clinician.

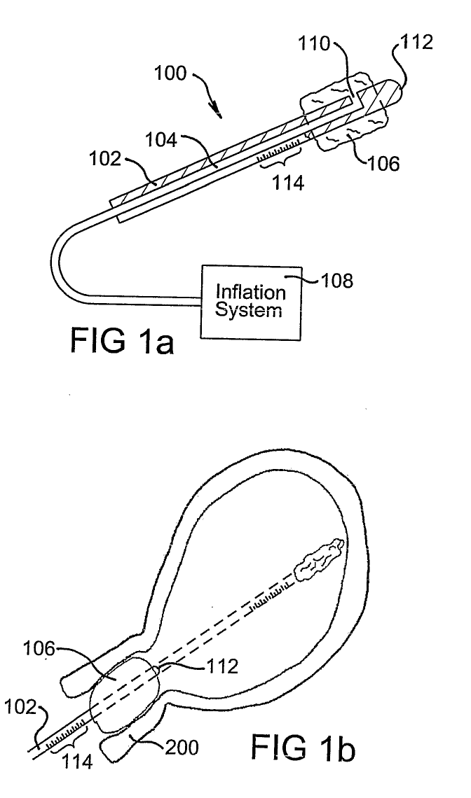

Referring to FIG. 1a, there is shown a device 100 for enlarging the cervical canal according to an embodiment of the invention. An elongate member 102 has a first lumen 104 extending therein. The first lumen is in communication toward its distal end with inflatable body 106 via one or more channels 110 which permit fluid flow from the lumen into the inflatable body. The inflatable body is shown in a deflated state, wrapped around or pressed onto the elongate member, thereby reducing the overall diameter of the device prior to insertion into the cervical canal. Where a plurality of channels 110 are provided, it is preferable that they are distributed evenly around the lumen to achieve substantially even inflation of the inflatable body.

The inflatable body 106 is preferably manufactured from a biocompatible polymer which is inflatable to withstand pressures of up to, for example, 100 psi although inflatable bodies with different pressure ratings may be provided such as, for example, 70 psi, 80 psi, 90 psi, 110 psi and 120 psi. Suitable polymers may include but are not limited to PET, nylon, medical grade plastic or PVC, elastomers and non-elastomeric materials.

The pressure rating of the inflatable member may correspond with or be accompanied by an inflation size rating where the inflation size rating is indicative of a maximum diameter to which the inflatable body will increase during inflation. Suitable inflation size ratings may correspond to balloon diameters of, for example, 6 mm, 8 mm, 10 mm, 12 mm, 14 mm, 16 mm, 18 mm and 20 mm. Preferably, the inflatable body is inextensible beyond a predetermined maximum inflation size to avoid over dilation of the cervical canal.

Preferably, the inflatable body has a substantially cylindrical shape. However, the inflatable body may be provided in a range of different shapes such as conical, hourglass or oblong, or cylindrical with curved ends. Various lengths may also be accommodated. Without limiting the possible range of lengths contemplated, the length of the inflatable body may range from e.g. 40 mm to 60 mm. Thus, a physician may select a device configuration with the inflatable body having a length, shape and inflation size rating which is appropriate for the subject being treated.

Selection of an appropriate device configuration is preferably based on an assessment of the subject's anatomy. In particular, the selection may be based on the anticipated maximum dilated cervix diameter which is required for a procedure, and/or the maximum dilation attainable in the subject without causing unnecessary cervical tearing and/or trauma.

The inflatable body may be attached to the elongate member using any suitable means. For example, it may be glued, welded or heat bonded to the elongate member, and wrapped around the shaft of the elongate member before being sterilised and packaged for distribution and use. Thus, the device is preferably disposable and amenable to use once only although re-sterilisable, re-usable devices may be suitable in some scenarios and are within the scope of the present invention.

The inflatable body may also include surface texture contours or other irregular surfacing which minimises or prevents the inflatable body from shifting inside the canal during inflation. The surface texture or contours may include an annular ring, a ridge, bump or texture on the external surface of the inflatable body to increase the friction between the inflatable body and the cervical tissue to help maintain its position while inflated.

The elongate member is sufficiently flexible that it may, where necessary, conform to the curvature of the subject's cervix and uterus. The elongate member may also possess sufficient rigidity that it can be inserted into a cervix, which is untreated by device or medication, without use of a guidewire or a sheath. It has a blunt distal tip and may be manufactured from a thin extrusion of flexible polymer such as rubber or plastic similar to other medical catheters. The inflatable body and/or elongate member may also be coated or treated with a lubricant to assist with insertion of the inflatable body into the cervical canal.

Preferably, the distal tip 112 of the device is rounded and smooth so that, in the event that the tip contacts the uterine wall during positioning of the device or sounding of the uterus, perforation is avoided. The distal tip may also include a coating which has a low coefficient of friction to encourage the tip to slide along the uterine wall when contact is made, although this feature may be omitted in embodiments used to sound the uterus prior to dilating the cervix since controlled contact between the distal tip and the uterine wall is necessary during sounding to determine an accurate measurement of uterine depth.

Markings 114 may also be provided to perform uterine sounding in which the device, with the inflatable body in the uninflated or deflated state is inserted through the cervical canal with the distal tip 112 of the member projecting into the uterine cavity. FIG. 1b shows the device, in broken lines, being used to sound the uterus prior to inflation of the inflatable body. The clinician uses tactile cues, gently touching the distal tip of the elongate member to the uterine wall, e.g. at the uterine fundus (the upper limit of the uterus) and a measurement is taken from the marking on the elongate member which is identified just exterior to the external cervical os.

The device may then be retracted, to position the inflatable body within the cervical canal. The clinician uses tactile cues felt through the device to determine when the inflatable body is positioned within the cervical canal and the inflatable body is then inflated using inflation system 108 and the cervix is dilated. At this stage, or prior to inflation, a second reading may be made using the markings. This can be used to ascertain the length of the cervical canal, by calculating the difference between the first and second depth readings.

At a proximal end the first lumen is in communication with an inflation system 108. The inflation system may be manually operable and controllable or it may be automated. For example, inflation system 108 may include a plunger and barrel arrangement such as, for example, a syringe manually operated by a clinician. Thus, when the elongate member and inflatable body are in position within the cervical canal, the clinician slowly advances the plunger within the barrel to force fluid into the lumen and inflate the inflatable body, slowly and gradually increasing the diameter of the cervical canal. Alternatively, inflation system 108 may consist of motor-driven pump which drives fluid into the lumen to inflate the inflatable body. Where a motor-driven pump is employed, the pump is preferably operated under the control of an automated control system programmed to deliver a slowly increasing pressure gradient to avoid sudden inflation which could cause unnecessary trauma and tissue damage.

Preferably, the automated control system receives inputs from sensors, e.g. pressure transducers, chemical sensors, and the like, to monitor parameters within the first lumen (and hence the inflatable body) and other lumens in the device. Where a control system is used to control operation of a motor-driven pump, the control system is preferably programmed with overriding safety routines which can terminate pressure increases during inflation in response to pressures detected in the lumen which exceed a threshold level. It may also operate a valve or other pressure release mechanism to release pressure from the lumen when a high pressure, capable of causing tissue damage or rupturing the inflatable body is detected.

The control system may be programmable with various pressure limits or thresholds for alarming or issuing other warning signals to the operator and for performing overriding safety routines. FIG. 1b shows, in solid lines, the device of FIG. 1a inflated inside a cervical canal 200.

The control system may also control injection of fluid into the uterine cavity via a second lumen in the device, and/or cycling of fluid into and out of the uterus. This can be useful to achieve amnio-infusion (i.e. flushing of the uterus with e.g. intravenous fluid). Pressure and other sensors for monitoring e.g. intra-uterine pressure, pH, etc are contemplated for use during procedures such as infusion of the uterus and hydrodilation of the fallopian tubes.

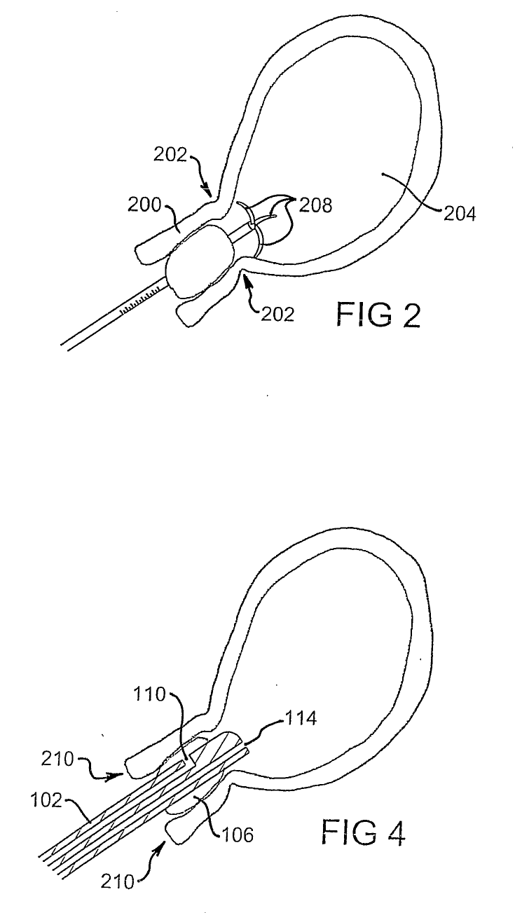

Turning now to FIG. 2, another embodiment of the present invention is shown which, in addition to the elongate member, lumen and inflatable body of FIGS. 1a and 1b, includes flexible projections 208 located on the elongate member just distal of the distal end of the inflatable body. The projections may take any suitable form and are gently outwardly biased. The projections may take the form of flexible, collapsible prongs as illustrated. Without wishing to limit the possible embodiments or combinations, there may be 3 projections, as illustrated, or 2 projections, or 4 or more projections.

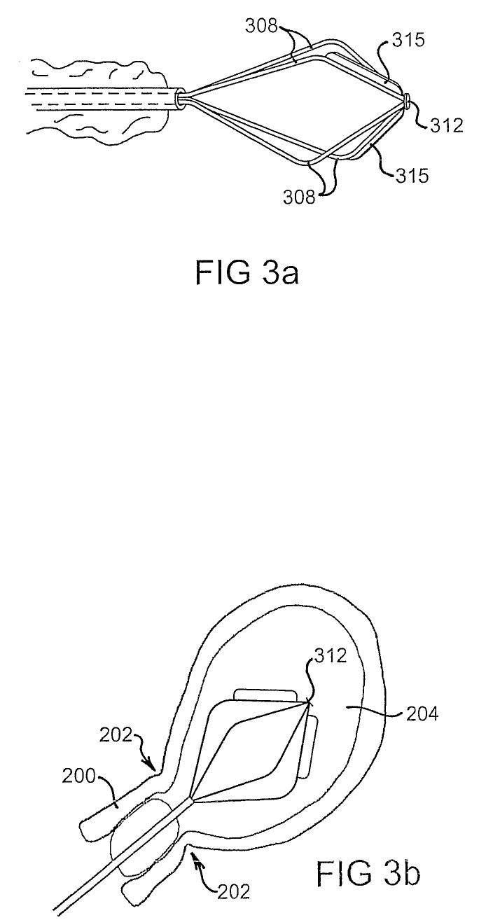

An alternative embodiment is illustrated in FIGS. 3a and 3b. These figures show the end portion of the device with the projections in the expanded configuration. In this embodiment, projections 308 are provided in the form of a collapsible framework terminating in a traumatic distal tip 312. Although the framework illustrated is expandable in three dimensions, it is to be understood that a simpler framework provided in a single plane could be similarly effective. Such a framework may have a diamond or suchlike shape expanding in one plane, or may be in the form of an expandable loop having no corner portions. The framework, in its expanded form may have a maximum cross section of e.g. 2 to 5 cm, although a maximum cross section of 3 to 4 cm is considered to be optimum for most female subjects.

The embodiment illustrated in FIGS. 3a and 3b also shows sampling edges 315 on two of the frame members. The sampling edges are adapted for removal of cells e.g. during biopsy, debris or other material from the wall of e.g. the uterus. The sampling edge may be provided on only one of the projections, or on all of the projections. Additionally, the sampling edge may extend along only a portion of the projection, or along substantially the entire length of the projection. Further, the sampling edge may be biased so as to facilitate removal of cells when the device is rotated e.g. in a clockwise direction while in situ, while substantially precluding removal of cells when the device is rotated in a counter-clockwise direction.

While the illustrated embodiment shows a straight sampling edge, it is to be understood that the edge could be curved, contoured or have other characteristics which facilitate the removal and/or adherence of cells and/or other matter from the uterine wall. Alternatively, the projections themselves, absent a sampling edge, could be used to scrape cells of the uterine wall.

The cells may adhere to the one or more projections or sampling edges for removal and pathology analysis. Alternatively, they may be sloughed off the uterine wall and extracted via a lumen in the device. Cell extraction in this manner may be further facilitated by filling the uterine cavity with liquid.

Prior to insertion of the device, the flexible prongs are collapsed and may extend along or distally of the elongate member to minimise the diameter of the device during insertion. Preferably, the distal region of the device has a maximum collapsed diameter, during insertion of 2-3 mm although collapsed diameters of 4 mm, 4.5 mm or 5 mm may be suitable for some subjects.

During insertion, the projections pass through the cervical canal in a collapsed state, without substantially increasing the resistance to insertion. While collapsed, the projections may give the distal end of the device an arrowhead-like appearance and may therefore have the effect of streamlining the device during insertion. The projections pass through the canal, past the internal cervical os and just into the uterine cavity 204 where, upon exiting the confines of the cervical canal 200, they adopt an outwardly biased state. A slight retraction of the elongate member will encounter a small resistance as the expanded projections abut the uterine wall around the internal cervical os 202, indicating to the clinician that the inflatable member is located within the cervical canal, and that inflation of the inflatable body to dilate the cervix may begin.

The collapsible projections are arranged relative to the inflatable body so as act as a guide to indicate to the administering clinician when the device has been located with the inflatable body positioned within the cervical canal. In various embodiments the one or more projections may also be used for uterine sounding. In the absence of one or more collapsible projections, the clinician would rely more heavily on tactile cues to determine when the inflatable body is within the cervical canal. This may increase the possibility of the inflatable body extending slightly past the internal or external cervical os during use. In these circumstances dilatation may be less efficient, or dilatation of an area which should not be dilated may occur, such as a false passage. This can prevent proper dilatation and/or result in perforation.

In addition, the projections may be used to slough cells of the uterine wall or, when the uterine has been fluid filled, to agitate the fluid to encourage cells to detach from the endometrial surface. The detached cells can then be withdrawn from the uterus through a second lumen in the elongate member and taken for pathology analysis or the like to diagnose the presence of a disease state or condition. Such cell samples may have particular utility in screening for cancer. Utilisation of the prongs to agitate fluid within the uterus could be combined with cycling of fluid within the uterus to encourage cell uptake.

To remove the device after the inflatable body has been deflated, a slightly stronger retraction force is applied to the elongate member to draw it out of the cervical canal. For the embodiment illustrated in FIG. 2, this will flex the projections distally, causing them to lie along the elongate member extending toward the distal tip, adopting an arrowhead appearance in the opposite direction. For the embodiment illustrated in FIGS. 3a and 3b, this will cause the framework to collapse for withdrawal of the device from the canal. This may cause slight discomfort for the subject although in various embodiments the projections are intended to be sufficiently flexible or flaccid that during retraction, they cause little or no tissue damage. In other embodiments, application of e.g. an electromagnetic field or a change in temperature or activation of an actuator may be deployed to controllably collapse the one or more projections prior to withdrawal of the device.

FIG. 4 shows a sectional view of a further embodiment of the present invention, in which a second lumen 114 is provided within the elongate member 102. Whilst the first lumen 104 delivers fluid to the inflatable body 106, second lumen 114 extends all the way through the elongate member and provides intra-uterine access from outside the external cervical os 210, while the device is in situ and the cervix is being dilated. In this embodiment, the device may also include means such as a tap, luer or other inlet for introducing a fluid such as a contrast die, saline, antibiotic, coagulant, therapeutic agent, gas or other liquid into the uterus. In addition, the device may include an outlet and be adapted to withdraw a fluid from the uterine cavity via the second lumen for the purpose of removing the fluid introduced to the uterus or removing any other fluid accumulated within the uterine cavity by other means, e.g. infection.

This enables the clinician to perform hydrodilation of the fallopian tube(s), a diagnostic test for determining fallopian tube patency. Alternatively, the second lumen may be used for instrumentation of the uterus during cervical dilation. By introducing fluid or an instrument in this way, a seal can be maintained within the cervical canal, substantially preventing leakage of fluid from the uterus and out of the cervical canal. A third lumen may be provided in the elongate member through which a second fluid may be introduced, or through which an instrument may be introduced into the cervix simultaneously with a fluid or instrumentation in the second lumen, while the device is in situ and the cervix is being dilated. The second an/or third lumens may also be used to deliver an implant such as a stent, staple or suture to the cervical canal or uterus.

Whilst the present invention has been described by reference to specific examples, it is to be understood that these embodiments are examples only and that other variations may be provided within the scope of the invention as described herein and within the scope of the claims appended hereto. For example, the distal tip of the elongate member protrude past the distal end of the inflatable body and project into the uterine cavity when the inflatable body is inflated within the cervical canal. This may be useful for sounding the uterus during cervical dilation, or for delivering fluids into deeper regions of the uterus while the cervix is dilated. It may also be useful for localised treatment of regions of the uterine wall by positioning the distal tip of the lumen near the region to be treated, and delivering fluid and/or an instrument through a second or third lumen in the elongate member. This arrangement is also useful for withdrawing e.g. cell samples from the uterine wall, with or without instrumentation. Using the device, discrete cell or tissue sample may be taken at a number of different locations around the uterus, and then withdrawn through the device during canal dilation.

The distal tip of the device may contact a wall of the uterus, such as the uterine fundus (the upper limit of the uterus), to perform sounding of the uterine cavity while the inflatable body is located substantially within the cervical canal. Sounding may be performed with the inflatable body in an inflated, uninflated or deflated state. In a variation of this embodiment, the inflatable body is movable, within limits, along the elongate member. This enables the uterus to be sounded while the inflated body is firmly placed within the cervical canal.

It is to be understood that various modifications, additions and/or alterations may be made to the parts previously described without departing from the ambit of the present invention as defined in the claims appended hereto.

1. A device for enlarging a canal of a human or animal subject, the device comprising:
an elongate member having a first lumen extending therein; and
an inflatable body located toward a distal end of the elongate member and deliverable, in an uninflated or deflated state, to a location substantially within the canal prior to inflation;
wherein the first lumen is in communication with the inflatable body; and
wherein, in use, the first lumen is in communication, toward a proximal end, with an inflation system for delivering fluid to inflate the inflatable body and thereby enlarging the canal.

2. A device according to claim 1, wherein the canal is the cervical canal of a subject.

3. A device according to claim 2 further comprising markings along a region of the elongate member extending outside the external cervical os during use of the device, the markings indicating a depth of insertion of the member into the cervical canal and/or uterine cavity.

4. A device according to claim 1, further comprising one or more collapsible projections located at or near the distal end of the elongate member, the one or more projections adapted to pass through the canal in a collapsed state and, upon passing into a distal chamber, assuming an expanded state.

5. A device according to claim 4 wherein the one or more collapsible projections are outwardly biased.

6. A device according to claim 4, wherein one or more of the collapsible projections comprises a sampling edge adapted for removal of cells or matter from within the distal chamber.

7. A device according to claim 6 wherein the sampling edge extends along a portion of the one or more collapsible projections.

8. A device according to claim 6 wherein the sampling edge extends along a substantial length of the one or more collapsible projections.

9. A device according to claim 4 wherein the collapsible projections comprise a collapsibly expandable framework.

10. A device according to claim 1 including means, located at or near the distal end of the elongate member, adapted to pass through the canal and to provide a tactile cue upon exiting a distal opening of the canal.

11. A device according to claim 1 wherein, prior to insertion, the diameter of the device in the region of the inflatable body is minimised by use of an adhesive retaining the inflatable body in a folded, wrapped, crumpled or otherwise minimised state, the adhesive being adapted to fail upon one or more of (a) inflation; (b) increase in temperature; (c) presence of moisture; and (d) presence of an electromagnetic field.

12. A device according to claim 1 wherein, during insertion into the canal, the overall diameter of the device in the region of the inflatable body is less than 5 mm.

13. A device according to claim 12 wherein, during insertion into the canal, the overall diameter of the device in the region of the inflatable body is 2 mm to 3 mm.

14. A device according claim 1, further comprising a lubricating coating on the inflatable body to aid insertion into the canal.

15. A device according claim 1 wherein the inflatable body has a substantially cylindrical, conical, hourglass, round or oblong shape when inflated.

16. A device according to claim 1 wherein the inflatable body conforms to natural contours of the canal when inflated.

17. A device according to claim 1, wherein the elongate member comprises a second lumen for providing access from outside the canal, to a chamber located distal of the canal, while the inflatable body is inflated within the canal.

18. A device according to claim 17 further comprising means for introducing a fluid into and withdrawing a fluid out of the chamber via the second lumen.

19. A device according claim 17 further comprising means for introducing an instrument into the chamber via the second lumen or a third lumen.

20. A device according to claim 1 wherein the inflatable body is manufactured from a material which is inextensible beyond a pre-defined shape and/or size.

21. A device according to claim 1 wherein a distal tip of the elongate member is adapted to make a traumatic contact with tissue of the canal or a distal chamber during insertion or the device and/or sounding of the chamber.

22. A device according to claim 1 wherein a distal tip of the elongate member comprises a soft and/or low friction coating to minimise risk of tissue trauma.

23. A device according to claim 1 wherein the inflated body is adapted to form a seal in the canal, thereby substantially precluding leakage of fluids from a distal chamber.

24. A device according to claim 2, for use in hydrodilation of a fallopian tube.

25. A device according to claim 1 further comprising an inflation system having a motor-driven pump under the control of an automated control system adapted to deliver gradually increasing pressure to the inflatable body.

26. A device according to claim 25 wherein the control system comprises one or more sensors to determine pressures within one or more of the inflatable body and a lumen within the elongate body, or a body chamber with which the lumen communicates.

27. A device according to claim 26 wherein the control system is adapted to, when a pressure above a predefined threshold is detected, cause release of pressure from within the inflatable body or within a lumen in communication with a body cavity.

28. A device according to claim 1 wherein the inflatable body is movable along a length of the elongate member.

29. A device according to claim 2, wherein the elongate member projects past the distal end of the inflatable body, such that the distal tip of the elongate member is capable of contacting a wall of the uterus while the inflatable body is located substantially within the cervical canal.

30. A method for increasing the diameter of a canal of a human or animal subject, comprising the steps of:
inserting a device, according to claim 1 into the canal, until the inflatable body is substantially within the canal in a deflated state; and
inflating the inflatable body by delivering fluid into the lumen, thereby increasing the diameter of the canal to a required level of dilatation.

31. A method for increasing the diameter of a canal according to claim 30, wherein the canal is the cervical canal of the subject and the method further comprises the step of sounding the uterus; wherein, prior to inflation of the inflatable body, the device is gently inserted until the distal tip of the device contacts a uterine wall, the device including markings along a length of the elongate member external to the external cervical os, and wherein a clinician takes a reading, using the markings, indicative of uterine depth.

32. A method of performing a procedure involving hydrodilation of a fallopian tube comprising the steps of claim 30, wherein inflation of the inflatable body forms a seal in a cervical canal of the subject; and further comprising the step of delivering a fluid, via a second lumen in the elongate member, to pressurise the subject's uterine cavity and achieve hydrodilation of one or both fallopian tubes.

33. A method according to claim 30 wherein the device is further utilised to extract cells from a cavity located distal of the canal.

34. A system for dilating a canal of a human or animal subject, the system comprising:
an enlarging device including an elongate member having a first lumen extending therein and in fluid communication with an inflatable body located toward a distal end of the elongate member, the inflatable body being deliverable, in an uninflated or deflated state, to a location substantially within the canal prior to inflation;
inflation means in fluid communication with the first lumen, for delivering fluid to inflate the inflatable body and achieve enlargement of the canal.

35. A system according to claim 34 wherein the device further comprises markings along a region of the elongate member extending outside the external opening of the canal during use of the device, the markings indicating a depth of insertion of the member into the canal and/or a chamber located distally of the canal.

36. A system according to claim 34 wherein the device further comprises one or more collapsible projections located at or near the distal end of the elongate member, the one or more projections adapted to pass through the canal in a collapsed state and, upon passing into a distal chamber, assuming an expanded state.

37. A system according to claim 36 wherein the one or more collapsible projections are outwardly biased.

38. A system according to claim 36 wherein one or more of the collapsible projections comprises a sampling edge adapted for removal of cells or matter from within a chamber distal located distally of the canal.

39. (canceled)

40. (canceled)
